(19) United States
(12) Patent Application Publication
(10) Pub. No.: US 2023/0148837 A1
(43) Pub. Date: May 18, 2023

(54) TIP COMPONENTS OF MULTI-CAMERA ENDOSCOPES

(71) Applicant: 270 SURGICAL LTD., Netanya (IL)

(72) Inventors: Golan SALMAN, Atlit (IL); Amram AIZENFELD, Kibbutz Ramot Menashe (IL); Suzy SALMAN, Tirat Carmel (IL)

(21) Appl. No.: 17/919,801

(22) PCT Filed: Apr. 8, 2021
(86) PCT No.: PCT/IL2021/050407

§ 371 Date: Oct. 19, 2022

Related U.S. Application Data
(60) Provisional application No. 63/012,569, filed on Apr. 20, 2020.

(51) Int. Cl. A61B 1/00; A61B 1/06

(52) U.S. Cl. CPC A61B 1/00096; A61B 1/00177; A61B 1/0615; A61B 1/0684; A61B 1/0011

(57) ABSTRACT

Provided herein is a tip component of a multi camera endoscope, the tip component accommodates a plurality of cameras configured to jointly provide a field-of-view (FOV) of at least about 270 degrees and a plurality of front illumination modules; wherein the tip component includes a plurality of window elements of sapphire glass soldered using a noble-metal, within respective openings on the housing thereof, wherein each of the cameras and illumination modules is positioned behind a respective window element. Further provided are methods of manufacturing the tip component and methods of using the same.

### TECHNICAL FIELD

The present disclosure relates generally to multi-camera endoscopes.

### BACKGROUND

An endoscope is a medical device used to image an anatomical site (e.g. a body cavity, a hollow organ). Unlike some other medical imaging devices, the endoscope is inserted into the anatomical site (e.g. through small incisions made on the skin of the patient). An endoscope can be employed not only to inspect an anatomical site and e.g. organs therein (and diagnose a medical condition in the anatomical site) but also as a visual aid in surgical procedures. Medical procedures involving endoscopy include laparoscopy, arthroscopy, cystoscopy, ureteroscopy, and hysterectomy.

### SUMMARY

Aspects of the disclosure, according to some embodiments thereof, relate to multi-camera endoscopes. More specifically, but not exclusively, aspects of the disclosure, according to some embodiments thereof, relate to tip components of multi-camera endoscopes and methods of manufacture thereof.

Thus, according to an aspect of some embodiments, there is provided an elongated shaft for a multi-camera endoscope. The shaft includes a shaft body and a tip component mounted on a shaft distal portion (of the shaft body). The tip component includes, accommodated within a housing of the tip component:
A front camera, a first side-camera, and a second side-camera positioned distally to the first side-camera. The side-cameras face oppositely, or substantially oppositely. The cameras are configured to jointly provide a field-of-view (FOV) of at least about 270 degrees.
A plurality of front illumination modules, two first side-illumination modules, and two second side-illumination modules. The two first side-illumination modules are respectively proximally and distally positioned relative to the first side-camera. The two second side-illumination modules are respectively proximally and distally positioned relative to the second side-camera. The illumination modules are configured to jointly illuminate the FOV.

The tip component includes a plurality of window elements of sapphire glass, which are soldered, using a noble-metal as solder material, within respective holes on the housing. Each of the cameras and the illumination modules is positioned behind a respective window element from the plurality of window elements.

According to some embodiments of the shaft, the noble metal includes gold and/or platinum.

According to some embodiments of the shaft, rims of the holes includes stainless steel. The window elements are soldered onto the rims.

According to some embodiments of the shaft, each of the window elements is set on a respective support ledge extending from a rim of the respective hole. The support ledges of at least some of the window elements, associated with the illumination modules, are segmented, thereby allowing provision of increased illumination by the illumination modules.

According to some embodiments of the shaft, segments pertaining to each of the segmented support ledges together circumferentially constitute less than 50% of a perimeter of the respective rim.

According to some embodiments of the shaft, the segments pertaining to each segmented support ledge consist essentially of four segments at most.

According to some embodiments of the shaft, a diameter D of the tip component measures between about 2 millimeters and about 15 millimeters.

According to some embodiments of the shaft, a characteristic scale d_{F} of the window element of the front camera measures between about 20% and about 50% of the diameter D of the tip component.

According to some embodiments of the shaft, at least some of the window elements, and holes corresponding thereto, are round or substantially round.

According to some embodiments of the shaft, the plurality of front illumination modules includes three illumination modules. A front surface of the housing may be flat, or substantially flat, and includes the window element of the front camera and three additional window elements. Each of the additional window elements has positioned behind thereto one of the front illumination modules, respectively. A relation between the diameter D, the characteristic scale d_{F}, and a spacing y_{F} between the window element of the front camera and nearest window element from the additional window elements is given by y_{F}≈(D−3·d_{F})/4.

According to some embodiments, the characteristic scale d_{F} measures about 3.4 millimeters.

According to some embodiments, 0.1 mm≤d_{F}≤5 mm. According to some embodiments, 2 mm≤d_{F}≤5 mm. According to some embodiments, 3.2 mm≤d_{F}≤4.9 mm. According to some embodiments, 0.1 mm≤d_{F}≤1 mm. According to some embodiments, 0.1 mm≤d_{F}≤0.4 mm.

According to some embodiments, each of the additional window elements has a respective characteristic scale which is comparable to the characteristic scale d_{F}.

According to some embodiments of the shaft, respective diameters of the window elements of the first and second side-cameras measure between about 2.5 millimeters and about 5 millimeters. Respective diameters of the window elements of the first and second side-illumination modules measure between about 3.5 millimeters and about 5.5 millimeters. Respective distances between each of the window elements of the side-cameras and the two window elements of the illumination modules, which are adjacent thereto, measure between about 0.5 mm and about 1.5 mm.

According to some embodiments of the shaft, the window elements of the cameras are all comparable in size. The window elements of the side illumination modules are all comparable in size.

According to some embodiments of the method, characteristic scales of the window elements of the side-cameras measure between about 30% to about 120% of the characteristic scale of the window element of the front camera.

According to some embodiments of the shaft, the window elements of the side-cameras have a smaller characteristic scale than the window elements of the side-illumination modules.

According to some embodiments of the shaft, each of the window elements has a thickness measuring between about 0.2 millimeters and about 1 millimeter.

According to some embodiments of the shaft, the gold-soldering between each of the window elements and the rim of the respective hole fluidly seals a gap between the window element and the rim. The fluid-sealing provided by the gold-soldering is configured to withstand autoclave sterilization.

According to some embodiments of the shaft, a width w of the gap measures between about 0.02 millimeters and about 0.1 millimeters.

According to some embodiments of the shaft, optical axes of the cameras span a plane comprising a longitudinal axis of the elongated shaft.

According to some embodiments of the shaft, the optical axis of the second side-camera is perpendicular to the longitudinal axis of the elongated shaft. The optical of the first side-camera is tilted by up to 5 degrees relative to the optical axis of the second side-camera and towards the optical axis of the front camera.

According to some embodiments of the shaft, the housing includes a first part, a second part, and a third part. The first part includes the front surface, the front camera, and the plurality of front illumination modules. The second part include a cover section, including the holes of the first side-camera and first side-illumination modules, and the first side-camera and first side-illumination units. The third part includes a hull, the second side-camera and second side-illumination units. The hull is open on a distal end thereof and a proximal end thereof, and includes an elongated opening on a first side-surface thereof, on which the cover section is configured to be fitted, and, on second side-surface thereof, the holes of the second side-camera and the second side-illumination modules. The hull is configured to accommodate the cameras and the illumination modules.

According to some embodiments of the shaft, each of the first part and second part is soldered, welded, and/or glued to the third part.

According to some embodiments of the shaft, each of the cameras is mounted on a respective printed circuit board (PCB).

According to some embodiments of the shaft, each of the illumination modules includes one or more light-emitting diodes (LEDs).

According to an aspect of some embodiments, there is provided a method for affixing windows on a shaft of a multi-camera endoscope. The method includes:
Providing a housing of a tip component of a shaft of a multi-camera endoscope. The housing includes at least a pair of holes arrangements. Each of the holes arrangements includes at least two holes for a window element for a camera and for at least one window element for at least one illumination module.
For each of the holes:
   Fitting a respective window element on a support ledge extending centrally from a rim of the hole.
   Soldering the window element onto the rim of the hole, thereby affixing the window element onto the housing.

The support ledges of at least some of the illumination module window elements are segmented, thereby allowing provision of increased illumination by the illumination modules.

According to some embodiments of the method, at least some of the window elements comprise sapphire glass and at least some of the window elements are soldered onto the rim of the respective hole using solder material comprising a noble metal.

According to some embodiments of the method, the noble metal comprises gold and/or platinum.

According to some embodiments of the method, segments pertaining to each of the segmented support ledges together circumferentially constitute less than 50% of a perimeter of the respective rim.

According to some embodiments of the method, the segments pertaining to each of the segmented support ledges consist of four segments at most.

According to some embodiments of the method, the soldering fluidly seals gaps between the window elements and the rims of the holes and is configured to withstand autoclave sterilization.

According to some embodiments of the method, each of the illumination modules includes one or more LEDs.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In the figures:

FIG. 1 is a schematic, perspective view of an endoscope, according to some embodiments;

FIGS. 2A and 2B are schematic, perspective side-views of a distal section of a shaft of an endoscope, which is a specific embodiment of the endoscope of FIG. 1;

FIG. 2C is a schematic, cross-sectional view of the distal section of the shaft of FIG. 2A, according to some embodiments;

FIG. 3 is a schematic, exploded view of a shaft distal section of an endoscope, which is a specific embodiment of the endoscope of FIG. 2A;

FIG. 4A is schematic, exploded view of a first part of a tip component of the shaft distal section of FIG. 3, according to some embodiments;

FIG. 4B is a schematic, exploded view of a second part of the tip component of the shaft distal section of FIG. 3, according to some embodiments;

FIG. 4C is a schematic, exploded view of a third part of the tip component of the shaft distal section of FIG. 3, according to some embodiments;

FIG. 5 is a schematic, cross-sectional view of the second part of FIG. 4B, according to some embodiments;

FIG. 6A is a schematic, perspective view of a hull, which forms part of a second part of a tip component of an endoscope, which is a specific embodiment of the endoscope of FIG. 3;

FIG. 6B is a schematic, side-view of the hull of FIG. 6A, according to some embodiments;

FIG. 7 is a schematic cross-sectional view of a window element mounted within a side-hole on a tip component of an endoscope, which is a specific embodiment of the endoscope of FIG. 2A;

FIG. 8 is a schematic, perspective view of a side-surface of a tip component of a specific embodiment of the shaft of FIG. 2A, the side-surface including a hole with full circumferential support for a window element;

FIG. 9 is a schematic, perspective view of a side-surface of a tip component of a specific embodiment of the shaft of FIG. 3, the side-surface including a hole with partial circumferential support for a window element;

FIG. 10 is a schematic, perspective, semi-transparent view of a hull of a second part of the tip component, which is a specific embodiment of the tip component of FIG. 9, according to some embodiments;

FIG. 11A schematically depicts a front holes arrangement on the tip component of the endoscope of FIG. 2A, according to some embodiments;

FIGS. 11B and 11C schematically depict a first side-holes arrangement and a second side-holes arrangement, respectively, on the tip component of the endoscope of FIG. 2A, according to some embodiments; and

FIG. 12 is a schematic, cross-sectional view of a first side-section of the endoscope of FIG. 2A, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 99% and 101% of the given value. In such embodiments, for example, the statement “the length of the element is equal to about 1 millimeter” is equivalent to the statement “the length of the element is between 0.99 millimeters and 1.01 millimeters”.

As used herein, according to some embodiments, the terms “substantially” and “about” may be interchangeable.

As used herein, a first quantity and a second quantity may be said to be “comparable” when the first quantity is about equal to the second quantity. Similarly, a first parameter and a second parameter may be said to be “comparable” (in magnitude) when a magnitude of the first parameter is about equal to a magnitude of the first parameter.

For ease of description, in some of the figures a three-dimensional cartesian coordinate system (with orthogonal axes x, y, and z) is introduced. It is noted that the orientation of the coordinate system relative to a depicted object may vary from one figure to another. Further, the symbol ⊙ may be used to represent an axis pointing “out of the page”, while the symbol ⊗ may be used to represent an axis pointing “into the page”.

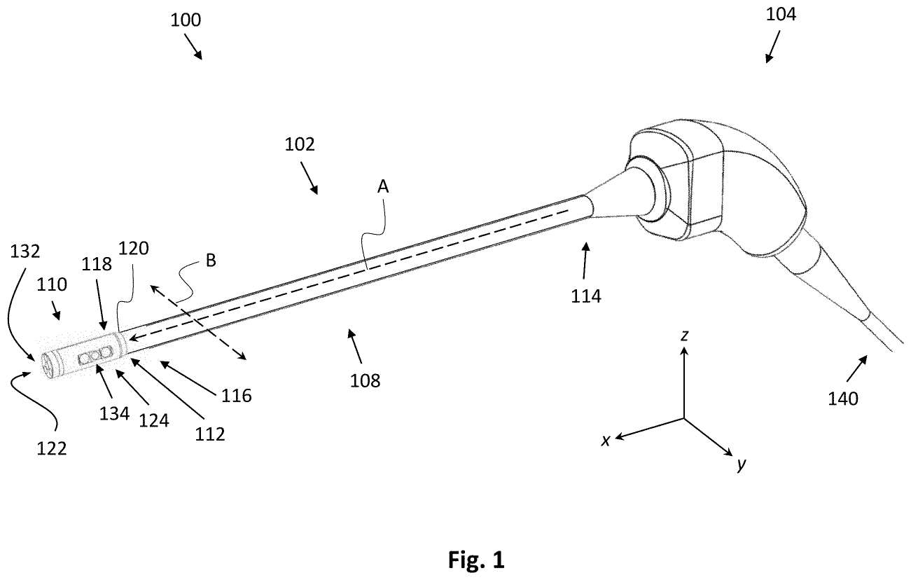

FIG. 1 schematically depicts an endoscope 100, according to some embodiments. Endoscope 100 includes an elongated shaft 102, configured to be inserted into an anatomical site (e.g. an anatomical cavity), and a handle 104, configured to be held by a user (e.g. a surgeon) of endoscope 100 and to facilitate guiding and manipulation of shaft 102 (particularly the distal section thereof) within the anatomical site. Shaft 102 further includes an (elongated) shaft body 108 and a (distal) tip component 110 mounted on a shaft body distal end 112. A shaft body proximal portion 114 is connected to, or configured to be connected to, handle 104, thereby mounting shaft 102 on handle 104.

According to some embodiments, each of shaft body 108 and tip component 110 may have a round or substantially round transverse cross-section. According to some embodiments, tip component 110 may be of a greater diameter than shaft 102 or at least a shaft body distal portion 116 (which includes shaft body distal end 112), as described in PCT application publication No. WO2019035118 to A. Levy et al., which is incorporated herein by reference in its entirety. According to some such embodiments, a tip proximal portion 118 (i.e. a proximal portion of tip component 110) may be tapered (i.e. incline in the proximal direction), terminating in a rim 120.

According to some embodiments, shaft 102 may have a diameter measuring between about 2 millimeters and about 15 millimeters. Tip component 110 may measure between about 1 millimeter and about 25 millimeters in length. According to some embodiments, tip component 110 may have a narrower diameter than the rest of shaft 102.

According to some embodiments, tip component 110 may be permanently attached to shaft body 108, e.g. using an adhesive or by soldering. Alternatively, according to some embodiments tip component 110 may be detachably mounted on shaft body 108. For example, tip component 110 may be screwed onto shaft body 108. Tip component 110 includes a front surface 122, a first side-surface 124, and a second side-surface (not visible in FIG. 1), positioned oppositely to first side-surface 124. Front surface 122 includes a front windows arrangement 132. First side-surface 124 includes a first side-windows arrangement 134. Optionally, in embodiments including three cameras (as elaborated on below), the second side-surface includes a second side-windows arrangement. Each of the windows arrangements (i.e. front windows arrangement 132, first side-windows arrangement 134, and the second side-windows arrangement (when included)) may include a plurality of windows, as described below.

Tip component 110 includes at least two cameras and a plurality of illumination modules. Each of the illumination modules is associated with a respective camera from the at least two cameras. In particular, each camera may be associated with a plurality of illumination modules. Each camera, and the one or more illumination modules associated therewith, are positioned behind a respective windows arrangement from the windows arrangements (i.e. front windows arrangement 132, first side-windows arrangement 134, and the second side-windows arrangement (when included)) such that the camera is positioned behind a respective window (dedicated to the camera) from the respective windows arrangement and each of the one or more illumination modules is positioned behind a respective window (dedicated to the illumination module) from the respective windows arrangement.

According to some embodiments, a front camera of the at least two cameras may face along, or substantially along, the distal direction (as indicated by a dashed arrow A). According to some embodiments, wherein the at least two cameras include three cameras, a first side-camera may face transversely (along one of the two opposite transverse directions indicated by a dashed double-headed arrow B), or substantially transversely. That is, the first side-camera may point, or substantially point, along a radial direction, and away from first side-surface 124. A second side-camera may face oppositely, or substantially oppositely, to the first side-camera. According to some embodiments, the two side-cameras may be positioned such that they are not back-to-back.

According to some embodiments, each of the illumination modules includes one or more light emitting diodes (LEDs).

According to some embodiments, the illumination modules include the distal tips of respective optical fibers. According to some such embodiments, handle 104 may include one or more light sources connected to one or more optical fibers extending through handle 104 and shaft 102. The optical fibers are configured to guide the light produced by the light sources from handle 104 to tip component 110, wherefrom the guided light may be shone such as to illuminate the field-of-view of the cameras. According to some embodiments, the light sources may be external to handle 104, being positioned, for example, in a main control unit (not shown). In such embodiments, a cable, which includes one or more optical fibers, may be used to guide the light, generated by the light sources in the main section, to handle 104. According to some embodiments, the cable may be a multi-purpose cable, such as a utility cable 140 described below.

Handle 104 may include a user control interface (not shown) configured to allow a user to control endoscope 100 functions. In particular, the user control interface may be functionally associated with the cameras and the illumination modules. According to some embodiments, the user control interface may allow, for example, to control zoom, focus, record/stop recording, and/or freeze frame functions of the cameras and/or to adjust the intensity of light provided by the illumination modules collectively and/or individually. The user control interface may include one or more buttons, knobs, switches, a touch panel, and/or the like.

According to some embodiments, endoscope 100 may be (i) directly maneuvered by a user through the manipulation of handle 104, as well as (ii) indirectly maneuvered, via robotics, e.g. using a robotic arm or other suitable gripping means configured to allow manipulation of handle 104.

The main control unit may be functionally associated with endoscope 100 via utility cable 140. The main control unit may include electronic circuitry (e.g. one or more processors and memory components) configured to process (digital data) from the cameras, such as to display the captured images and video(s) on a monitor. In particular, the processing circuitry may be configured to process the digital data received from each of the cameras, such as to produce therefrom a combined video file/stream providing a continuous and consistent (seamless) panoramic view of an anatomical site wherein endoscope 100 is inserted.

According to some alternative embodiments, the main control unit may be functionally associated with endoscope 100 through wireless communication.

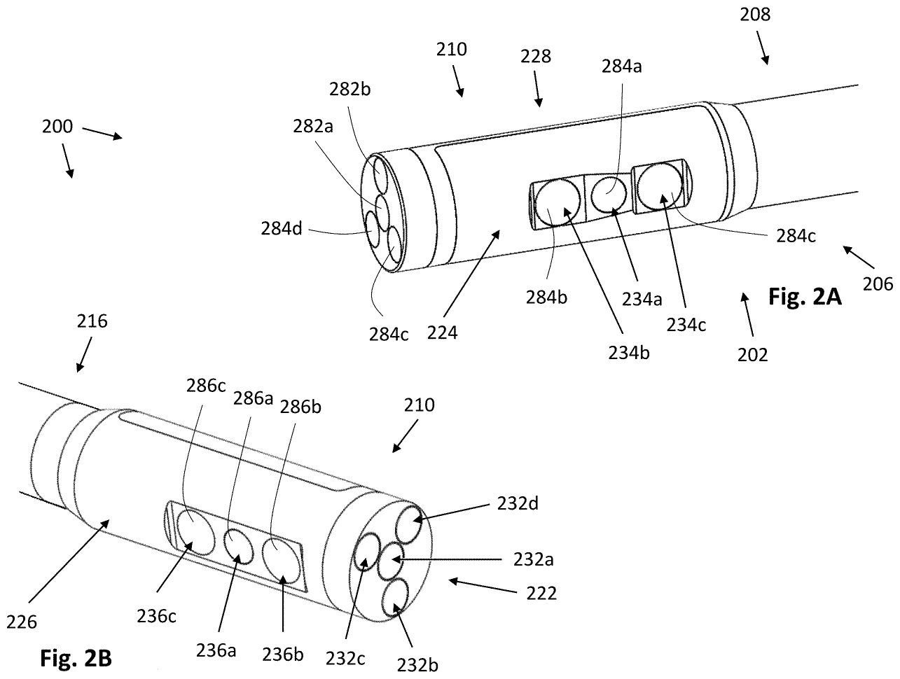

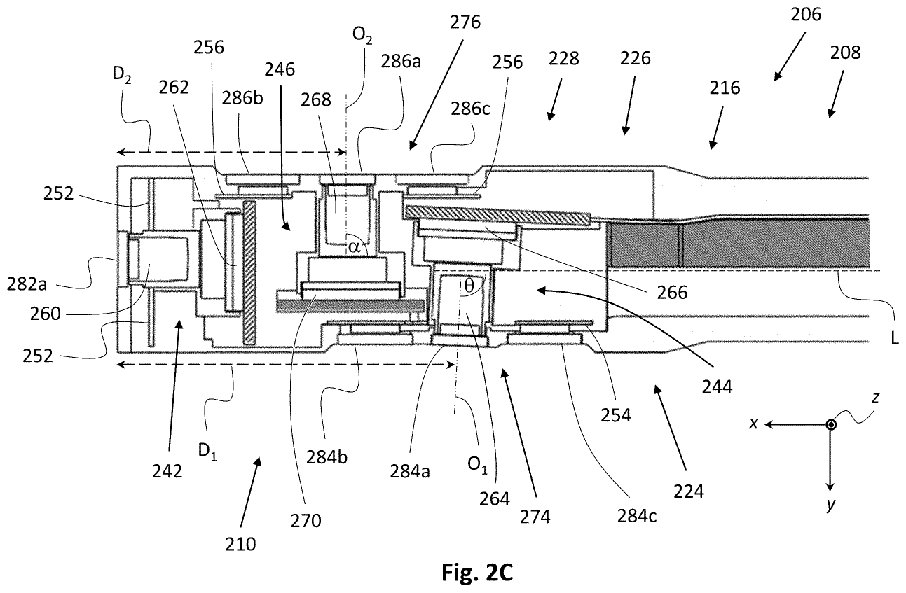

FIGS. 2A and 2B are schematic perspective side-views of a (shaft) distal section 206 of a shaft 202 of an endoscope 200, which is a specific embodiment of endoscope 100. Endoscope 200 includes shaft 202 and a handle (not shown), such as handle 104. Shaft 202 is a specific embodiment of shaft 102 and includes a shaft body 208 and a tip component 210, which are specific embodiments of shaft body 108 and tip component 110. Also indicated is a housing 228 of tip component 210, which is configured to accommodate internal components, such as cameras, illumination units, electronics, and so on. Shaft distal section 206 includes tip component 210 and a shaft body distal portion 216 of shaft body 208. Also indicated are a front surface 222, a first side-surface 224, and a second side-surface 226 of tip component 210.

Making reference also to FIG. 2C, FIG. 2C is a schematic, longitudinal cross-sectional view of shaft distal section 206, according to some embodiments. According to some embodiments, and as depicted in FIG. 2C, tip component 210 includes three cameras and three illumination units: a front camera 242, a first side-camera 244, a second side-camera 246, a front illumination unit 252, a first side-illumination unit 254, and a second side-illumination unit 256. According to some embodiments, front illumination unit 252 may include three illumination modules, while each of side-illumination units 254 and 256 may include two illumination modules.

Each of cameras 242, 244, and 246 includes a lens assembly and an image sensor: Front camera 242 includes a front lens assembly 260 and a front image sensor 262, first side-camera 244 includes a first side-lens assembly 264 and a first side-image sensor 266, and second side-camera 246 includes a second side-lens assembly 268 and a second side-image sensor 270. According to some embodiments, each of image sensors 262, 266, and 270 is a CMOS (complementary metal-oxide semiconductor) image sensor, but it will be understood that other options are possible. In particular, according to some alternative embodiments, one or more of the image sensors may be CCD (charge-coupled device) image sensors.

According to some embodiments, and as depicted in FIG. 2C, first side-camera 244 and second side-camera 246 are not positioned back-to-back. That is, first side-camera 244 is positioned within tip component 210 at a distance D₁ from front surface 222 of tip component 210, and second side-camera 246 is positioned at a distance D₂ from front surface 222, with D₂<D₁. According to some such embodiments, first side-camera 244 and second side-camera 246 may be positioned adjacently to one another along the longitudinal axis L of shaft 202, thereby saving space and helping to restrict the lateral dimensions (i.e. the diameter) of shaft distal section 206. The longitudinal axis L extends along the length of shaft 202 (and therefore in parallel to the x-axis). According to some such embodiments, the diameter of shaft distal section 206 may be smaller than about 11 millimeters. According to some embodiments, the distance D₁ may be between about 10 millimeters and about 27 millimeters, and the distance D₂ may be between about 10 millimeters and about 17 millimeters. According to some embodiments, the distance D₁ is between about 4 millimeters and about 15 millimeters, and the distance D₂ is between about 4 millimeters and about 15 millimeters, but at the same time D₂ may be smaller than D₁.

According to some embodiments, and as depicted in FIGS. 2A-2C, front lens assembly 260 may be embedded in or on front surface 222, such as to face forward along the direction defined by the positive x-axis, i.e. the distal direction. First side-lens assembly 264 may be embedded in or on first side-surface 224, such as to face sideways, along a direction, which as shown in FIG. 2C, is slightly tilted relative to the direction defined by the positive y-axis. That is, an optical axis O₁ of first side-lens assembly 264 may be slightly tilted (e.g. on a plane parallel to the xy-plane) relative to the y-axis (as indicated by an angle θ, defined by the optical axis O₁ and the longitudinal axis L, which is slightly greater than 90 degrees in FIG. 2C). As a non-limiting example, according to some embodiments, θ may be between 90.5 degrees and 95 degrees. Second side-lens assembly 268 may be embedded in or on second side-surface 226, such as to face sideways, along the direction defined by the negative y-axis. (So that an optical axis O₂ of second side-lens assembly 268 is parallel to they-axis, or, what is the same thing, perpendicular to the longitudinal axis L).

Also indicated is an angle α between optical axis O₂ and longitudinal axis L. According to some embodiments, not depicted in FIG. 2C, optical axis O₂ may be slightly tilted relative to longitudinal axis L. As a non-limiting example, according to some embodiments, α may be between 90.5 degrees and 95 degrees.

According to some embodiments, cameras 242, 244, and 246 are configured to provide, in combination, a continuous field-of-view (FOV) of at least about 270 degrees. More specifically, the horizontal FOV provided by the cameras may be at least about 270 degrees, wherein the horizontal plane may be parallel to the xy-plane. According to some such embodiments, the optical axes of cameras 242 (not shown), 244 (i.e. optical axis O₁), and 246 (i.e. optical axis O₂) all lie on plane parallel to the xy-plane. The positioning of the cameras within tip component 210 may be selected such as to minimize the space occupied by the cameras and reduce the diameter of tip component 210 and shaft distal section 206, while affording a continuous FOV at least about 270 degrees.

According to some alternative embodiments, not depicted in the figures, first side-camera 244 and second side-camera 246 are positioned back-to-back.

Each of cameras 242, 244, and 246, and illumination units 252, 254, and 256, may be functionally associated with electronic components (such as processors, amplifiers, discrete components), which may be mounted on one or more printed circuit boards (PCBs) and/or connected to one or more electrical wires. According to some embodiments, the PCBs may be foldable such as to allow compact accommodation of the cameras within tip component 210.

Referring again to FIGS. 2A and 2B, tip component 210 includes a front windows arrangement 232, a first side-windows arrangement 234, and a second side-windows arrangement 236. Front windows arrangement 232 may include a central window 232a, and three windows 232b, 232c, and 232d surrounding central window 232a. Front camera 242 may be positioned behind (i.e. proximally to) central window 232a (and is, thus, hidden from view in FIGS. 2A and 2B). The three illumination modules of front illumination unit 252 may be positioned behind windows 232b, 232c, and 232d, respectively (and are, thus, not visible in FIGS. 2A and 2B). According to some embodiments, each of the three illumination modules may include a plurality of LEDs, for example, two, three, or four LEDs which may be arranged in an array. According to some embodiments, the LEDs may emit light at the same wavelength. According to some alternative embodiments, different LEDs may emit light at different wavelengths, respectively. According to some embodiments, the windows may differ from one another in shape and/or in size. In particular, according to some embodiments, central window 232a may differ in shape and/or size from windows 232b, 232c, and 232d.

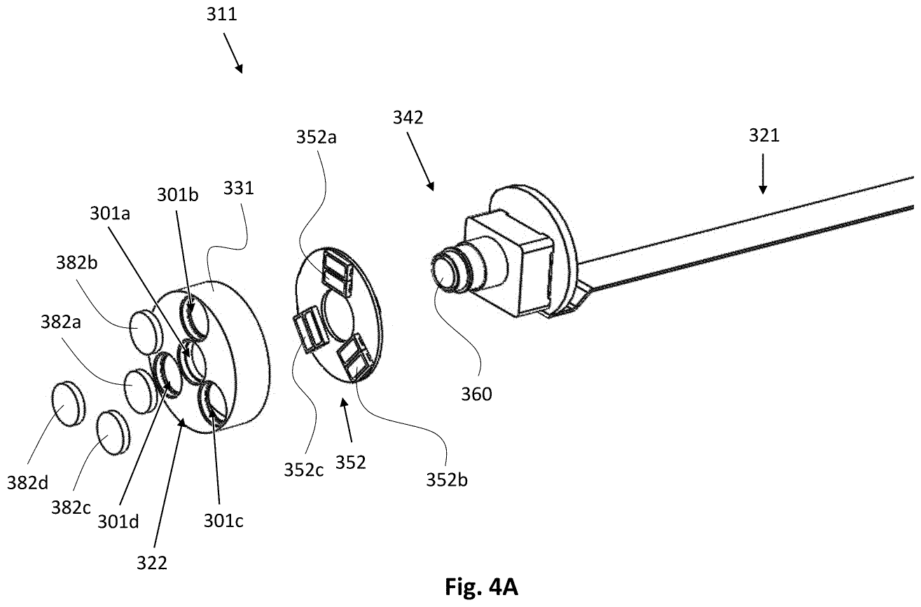

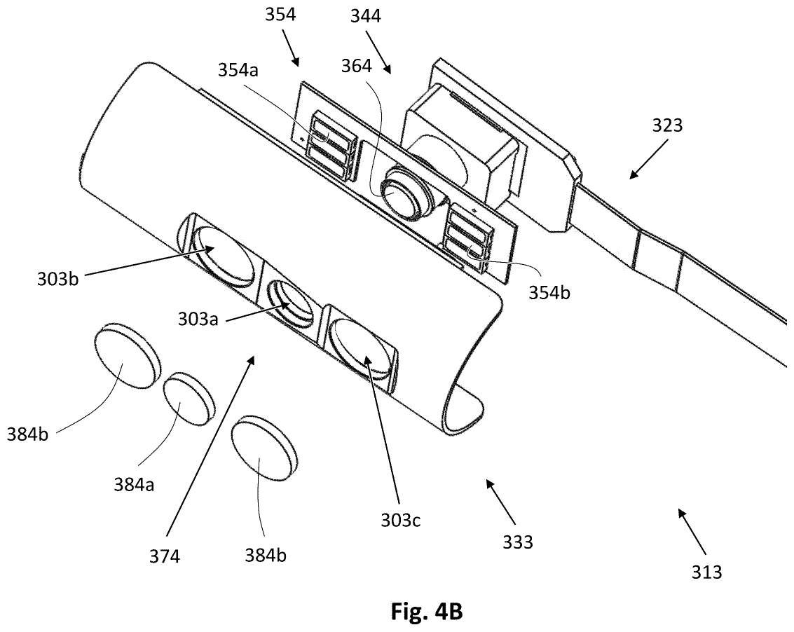

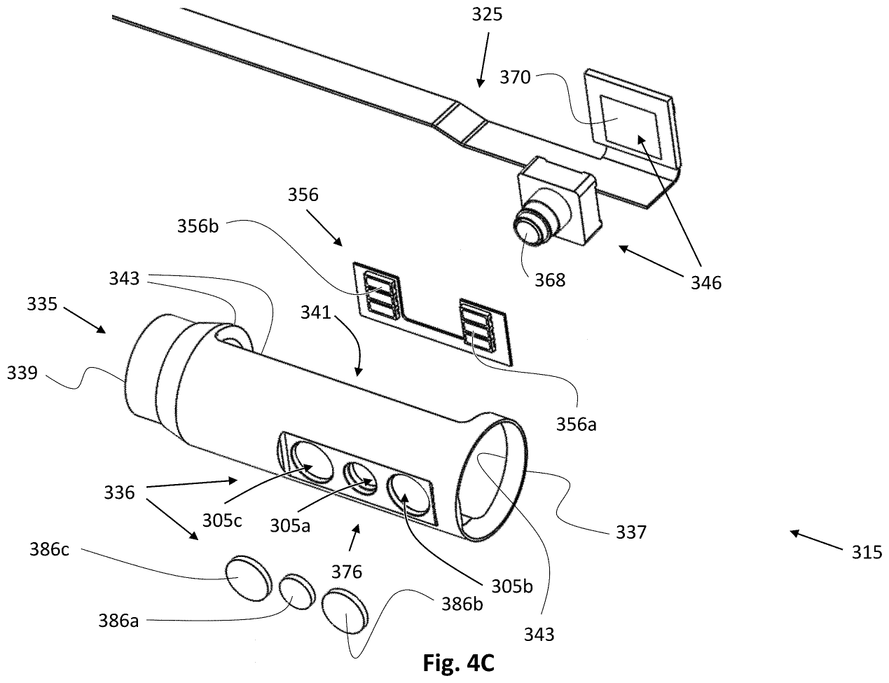

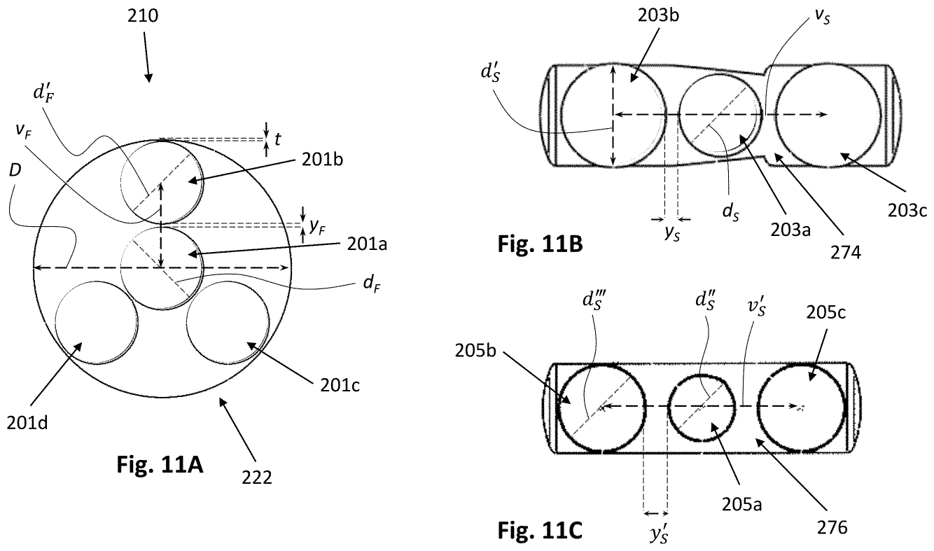

Front surface 222 includes a front holes arrangement 201 (i.e. a front arrangement of holes; shown in FIG. 11A). Each of the holes has set therein a respective window element (e.g. a windowpane) pertaining to front windows arrangement 232. Indicated are a central window element 282a, and window elements 282b, 282c, and 282d, pertaining to central window 232a, and windows 232b, 232c, and 232d, respectively. Each hole-window element pair constitutes a window (from one of the window arrangements). For example, a central front hole 201a (from front holes arrangement 201) and central window element 282a constitute central window 232a. Each of the window elements of front windows arrangement 232 is set within a corresponding hole from the front holes arrangement. For example, window element 282a is set within central front hole 201a. More specifically, each of the holes is shaped and dimensioned to have affixed therein (for example, by soldering) a corresponding window element, essentially as elaborated on below in the description of FIGS. 4A-4C.

First side-windows arrangement 234 may include a central window 234a and two windows 234b and 234c respectively positioned on opposite sides of central window 234a. First side-camera 244 may be positioned behind central window 234a. The two illumination modules of first side-illumination unit 254 may be positioned behind windows 234b and 234c, respectively (and are, thus, not visible in FIG. 2A). Each of the two illumination modules may include a plurality of light emitting diodes (LEDs, e.g. which may be arranged in an array). According to some embodiments, the windows may differ from one another in shape and/or in size. In particular, according to some embodiments, central window 234a may differ in shape and/or size from windows 234b and 234c.

First side-surface 224 includes a first side-holes arrangement 203 (shown in FIG. 11B). Each of the holes has set therein a respective window element pertaining to first side-windows arrangement 234. Indicated are a central window element 284a, and window elements 284b and 284c, pertaining to central window 234a, and windows 234b and 234c, respectively. More specifically, each of the holes is shaped and dimensioned to have affixed therein (for example, by soldering) a corresponding window element from first side-windows arrangement 234.

Second side-windows arrangement 236 may include a central window 236a and two windows 236b and 236c respectively positioned on opposite sides of central window 236a. Second side-camera 246 may be positioned behind central window 236a (and is, thus, hidden from view in FIG. 2B). The two illumination modules of second side-illumination unit 256 may be positioned behind windows 236b and 236c, respectively (and are, thus, not visible in FIG. 2B). Each of the two illumination modules may include a plurality of LEDs (e.g. arranged in an array). According to some embodiments, the windows may differ from one another in shape and/or in size. In particular, according to some embodiments, central window 236a may differ in shape and/or size from windows 236b and 236c.

Second side-surface 226 includes a second side-holes arrangement 205 (shown in FIG. 11C). Each of the holes has set therein a respective window element pertaining to second side-windows arrangement 236. Indicated are a central window element 286a, and window elements 286b and 286c, pertaining to central window 236a, and windows 236b and 236c, respectively. More specifically, each of the holes is shaped and dimensioned to have affixed therein (for example, by soldering) a corresponding window element from second side-windows arrangement 236.

The windows (i.e. the windows of window arrangements 232, 234, and 236) protect the cameras (i.e. cameras 242, 244, and 246) and the illumination units (i.e. illumination units 252, 254, and 256) from body fluids and debris during an endoscopy procedure. Further, shaft distal section 206 (and, in particular, tip component 210) may be fluidly sealed such as to prevent air/gas from entering therein during an endoscopy procedure and when undergoing cleaning. Air penetration may lead to the formation of a condensate on the lenses of the cameras and the inner surfaces of the windows, and thereby blur video and images obtained by the cameras. Moisture may lead to corrosion of the electrical components within shaft distal section 206, which may result in malfunctioning of the cameras and the illumination units. In order to prevent air penetration, an inert gas, such as nitrogen, may be pumped into shaft distal section 206, prior to the sealing thereof. The illumination modules (on the illumination units), which may include light sources, such as LEDs, are configured to illuminate the field-of-view of the cameras.

According to some embodiments, each window element (windowpane) in each of the window arrangements (i.e. window arrangements 232, 234, and 236) may be made of, or include, sapphire glass (synthetic sapphire). According to some embodiments, front surface 222 and each of side-surfaces 224, and 226 may be made of, or include, stainless steel. According to some embodiments, rims of the holes (of the holes arrangements) are made of, or include, stainless steel. According to some such embodiments, each of the window elements may be bonded to the rim of the hole wherein the window element is set, using solder material configured to strongly and permanently bond sapphire glass to stainless steel. According to some embodiments, the solder material is, or includes, a noble metal, such as, for example, gold, silver, and/or platinum, thereby helping to ensure the integrity of the bond over time. More specifically, each hole may be slightly larger than the corresponding window element, such that, when the window element is positioned within the hole, with the respective center points thereof coinciding, a gap is present between the window element and the rim of the hole. The gap is sufficiently wide to accommodate the solder material which bonds the window element to the rim (i.e. the solder material affixes the window element within the hole). In particular, the solder material and each hole-window element pair are configured such that the solder material fluidly seals the gap between the hole and the window element and remains intact under steam sterilization (e.g. autoclave sterilization).

According to some embodiments, wherein the window elements are made of sapphire glass and the side-surfaces are made of stainless steel, the thickness of the sapphire glass window elements is selected to be sufficiently thick to (i) maintain integrity during the soldering of the window elements to the holes, and (ii) not be deformed or fractured due to mechanical stresses which the stainless steel side-surfaces may exert on the window elements (e.g. due to thermal expansion or contraction).

According to some embodiments, first side-windows arrangement 234 may be set within a first side-niche 274 (indicated in FIG. 2C) within first side-surface 224. First side-niche 274 may form a shallow indentation on first side-surface 224. Similarly, according to some embodiments, second side-windows arrangement 236 may be set within a second side-niche 276 (indicated in FIG. 2C) within second side-surface 226. Second side-niche 276 may form a shallow indentation on second side-surface 226. Each of side-niches 274 and 276 may form a flat depression on the respective side-surface, thereby allowing to affix within holes in the depression flat windows elements, which may be easier to produce than concave window elements and may potentially be more durable. Thus, according to some embodiments, side-niches 274 and 276 form a flat depressions, and each of window elements 284a, 284b, and 284c and windows 286a, 286b, and 286c is flat.

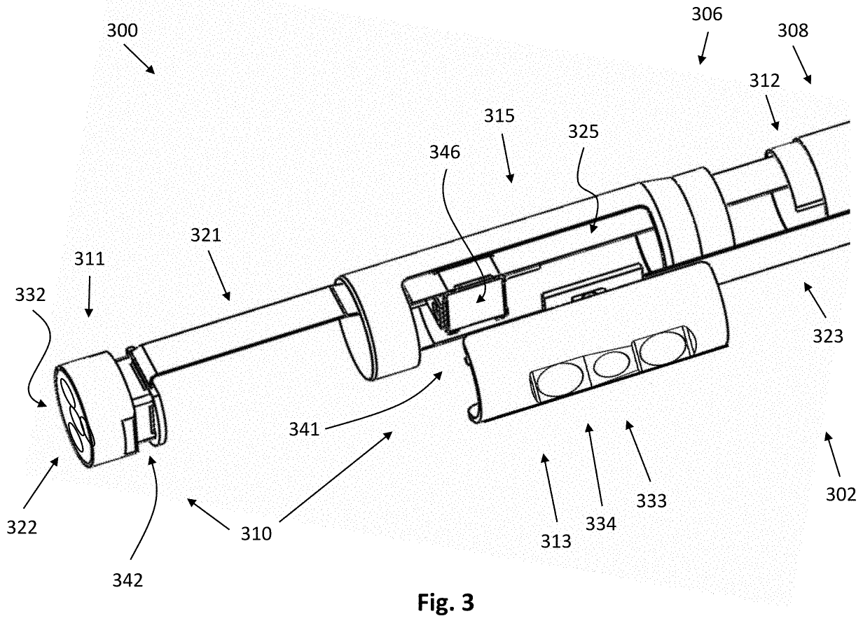

FIG. 3 provides a schematic, exploded view of a shaft distal section 306 of a shaft 302 of an endoscope 300, which is a specific embodiment of endoscope 200. In particular, shaft distal section 306 is a specific embodiment of shaft distal section 206 and includes a tip component 310 which is a specific embodiment of tip component 210. According to some embodiments, and as depicted in FIG. 3, tip component 310 is formed of three parts: a tip first part 311, a tip second part 313, and a tip third part 315. The three parts may be soldered, welded (e.g. laser welded), and/or glued onto one another, such as to fluidly seal tip component 310 and shaft distal section 306. Also indicated are a shaft body 308 (of shaft 302), which is a specific embodiment of shaft body 208, a front windows arrangement 332, which is a specific embodiment of front windows arrangement 232, and a first side-windows arrangement 334, which is a specific embodiment of first side-windows arrangement 234.

Referring also to FIGS. 4A-4C, FIG. 4A provides a schematic, exploded view of tip first part 311, according to some embodiments. FIG. 4B provides a schematic, exploded view of tip second part 313, according to some embodiments. FIG. 4C provides a schematic, exploded view of tip third part 315, according to some embodiments.

According to some embodiments, tip first part 311 includes a circumferential frame 331, a front surface 322, front windows arrangement 332, a front camera 342, a front illumination unit 352, and a first PCB 321. Front illumination unit 352 may include three front illumination modules: a front illumination module 352a, a front illumination module 352b, and a front illumination module 352c. Front windows arrangement 332 includes window elements 382 and a front holes arrangement 301. Indicated are window elements 382a, 382b, 382c, and 382d—of front windows arrangement 332—which correspond to front holes 301a, 301b, 301c, and 301d, respectively. Front surface 322, front camera 342, and front illumination unit 352 are specific embodiments of front surface 222, front camera 242, and front illumination unit 252, respectively. Circumferential frame 331 distally terminates in front surface 322. According to some embodiments, circumferential frame 331 may be characterized by a round or oval transverse cross-section.

According to some embodiments, tip second part 313 includes a cover section 333, a first side-niche 374, first side-windows arrangement 334, a first side-camera 344, a first side-illumination unit 354, and a second PCB 323. First side-illumination unit 354 may include two side-illumination modules: a side-illumination module 354a and a side-illumination module 354b. First side-windows arrangement 334 includes window elements 384 and a first side-holes arrangement 303. Indicated are window elements 384a, 384b, and 384c—of first side-windows arrangement 334—which correspond to first side-holes 303a, 303b, and 303c, respectively. First side-niche 374, first side-camera 344, and first side-illumination unit 354 are specific embodiments of first side-niche 274, first side-camera 244, and first side-illumination unit 254, respectively. The first side-windows arrangement is included in cover section 333, as elaborated on below.

According to some embodiments, tip third part 315 includes a hull 335, a second side-niche 376, a second side-windows arrangement 336, a second side-camera 346, a second side-illumination unit 356, and a third PCB 325. Second side-illumination unit 356 may include two side-illumination modules: a side-illumination module 356a and a side-illumination module 356b. Second side-windows arrangement 336 includes window elements 386 and a second side-holes arrangement 305. Indicated are window elements 386a, 386b, and 386c corresponding to second side-holes 305a, 305b, and 305c, respectively. Window elements 386a, 386b, and 386c form part of second side-windows arrangement 336, which is a specific embodiment of second side-windows arrangement 236. Second side-niche 376, second side-camera 346, and second side-illumination unit 356 are specific embodiments of second side-niche 276, second side-camera 246, and second side-illumination unit 256, respectively.

Hull 335 is dimensioned such as to accommodate (at least) cameras 242, 244, and, 246, illumination units 252, 254, and 256, and PCBs 321, 323, and 325. More specifically, hull 335 is hollow, being open on a hull distal end 337 (i.e. a distal end of hull 335) and on a hull proximal end 339. Circumferential frame 331 is configured to be fitted on hull distal end 337 (thereby mounting tip first part 311 on tip third part 315). Hull 335 further includes a side-opening 341 whereon cover section 333 is configured to be fitted (thereby mounting tip second part 313 on tip third part 315). Also indicated is a rim 343 of side-opening 341. Finally, hull proximal end 339 is configured to be fitted on a shaft body distal end 312 (i.e. the distal end of shaft body 308), thereby attaching tip component 310 to shaft body 308.

First PCB 321 may have front camera 342 mounted thereon, as well as electronic components related to the operation of front camera 342 and front illumination unit 352, such as electronic switches and/or amplifiers configured, for example, to switch on/off front camera 342 and to set an (illumination) intensity of front illumination unit 352. Second PCB 323 may have first side-camera 344 mounted thereon, as well as electronic components related to the operation of first side-camera 344 and first side-illumination unit 354. Third PCB 325 may have second side-camera 346 mounted thereon, as well as electronic components related to the operation of second side-camera 346 and second side-illumination unit 356.

According to some embodiments, one or more of PCBs 321, 323, and 325 may be foldable. For example, according to some embodiments and as depicted in FIGS. 4A and 4C, respectively, first PCB 321 and third PCB 325 are foldable.

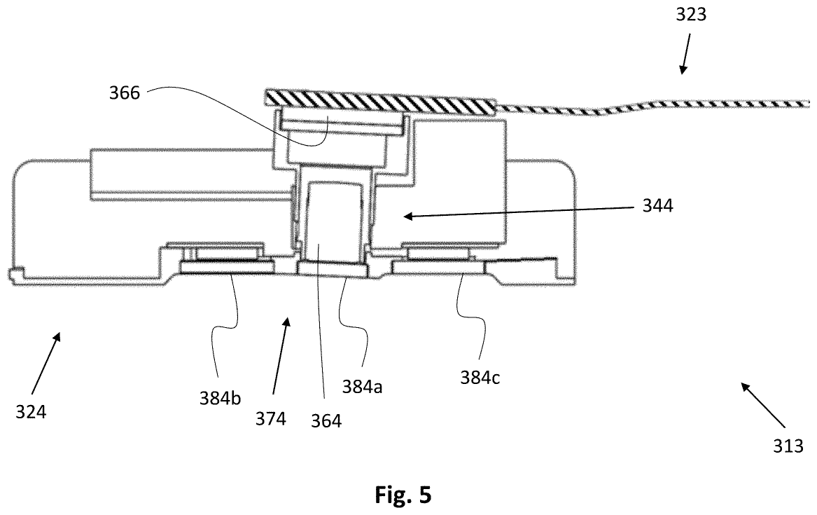

FIG. 5 provides a schematic cross-sectional view of tip second part 313, according to some embodiments. A first side-lens assembly 364 and a first side-image sensor 366 of first side-camera 344 are also indicated. First side-lens assembly 364 and first side-image sensor 366 are integrated within tip second part 313. Window elements 384 are shown positioned within first side-niche 374.

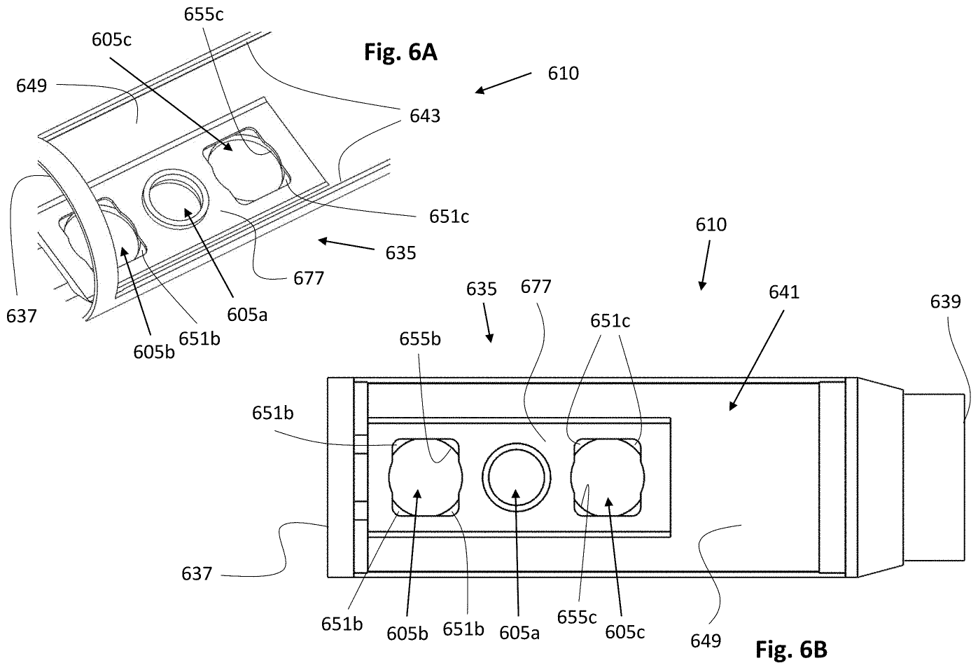

FIG. 6A provides a schematic, perspective view of a hull 635 of a tip component 610, according to some embodiments. FIG. 6B provides a side-view of hull 635, according to some embodiments. Tip component 610 is a specific embodiment of tip component 310. Hull 635 is a specific embodiment of hull 335 of tip third part 315 of endoscope 300. In both of FIGS. 6A and 6B, an inner surface 649 of hull 635, and a niche inner surface 677, are visible. Niche inner surface 677 is included in inner surface 649. Niche inner surface 677 constitutes the inner surface of the wall of the indentation defined by a second side-niche (not visible in FIGS. 6A and 6B), which is a specific embodiment of second side-niche 376. The second side-niche includes a (second) side-holes arrangement 605, which is a specific embodiment of second side-holes arrangement 305. According to some embodiments, and as depicted in FIGS. 6A and 6B, niche inner surface 677 may be flat. Also indicated are a hull distal end 637, a hull proximal end 639, a side-opening 641, and a rim 643 of side opening 641.

Side-holes arrangement 605 includes a central side-hole 605a and two side-holes 605b and 605c respectively positioned distally and proximally to central side-hole 605a. According to some embodiments, niche inner surface 677 includes corner surface-niches 651b (not all of which are marked), each of which forms a respective depression extending from a rim 655b of side-hole 605b. Corner surface-niches 651b are configured to accommodate corners of an illumination module, which is characterized by a height and/or width greater than the diameter of side-hole 605b. In particular, each of corner surface-niches 651b may constitute a respective region of reduced thickness of the wall of the indentation, defined by the second side-niche, as compared to the rest of the wall. Similarly, according to some embodiments, niche inner surface 677 includes corner surface-niches 651c (not all of which are marked) forming respective depressions extending from a rim 655c of side-hole 605c. Corner surface-niches 651c are configured to accommodate corners of an illumination module, which may be characterized by a height and/or width greater than the diameter of side-hole 605c. In particular, each of corner surface-niches 651c may constitute a respective region of reduced thickness of the wall of the indentation, defined by the second side-niche, as compared to the rest of the wall.

As used herein, according to some embodiments, the terms “corner niche” and “corner surface-niche” may be used interchangeably.

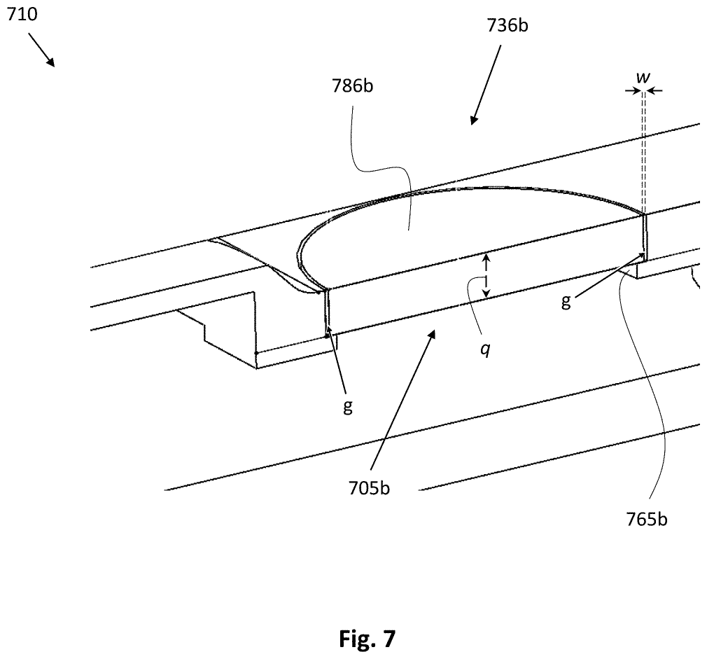

FIG. 7 provides a schematic cross-sectional view of a portion of a tip component 710, according to some embodiments. Tip component 710 is a specific embodiment of tip component 210. According to some embodiments, tip component 710 is a specific embodiment of tip component 310. According to some embodiments, tip component 710 is a specific embodiment of tip component 610. The cross-section is taken such as to bisect a window 736b of a (second) side-holes arrangement, which is a specific embodiment of window 236b (and according to some embodiments, a specific embodiment of the distalmost window from second side-windows arrangement 336 of tip component 310). Window 736b includes a side-hole 705b and a window element 786b soldered thereto. According to some embodiments, side-hole 705b and window element 786b are a specific embodiments of side-hole 305b and window element 386b, respectively.

Indicated is a gap g between a window element 786b and a rim of side-hole 705b. Also indicated is a width w of the gap g. When window element 786b is affixed within side-hole 705b, the gap g is filled with solder material, which bonds window element 786b (more precisely, the rim thereof) to a rim of side-hole 705b. According to some embodiments, the width w of the gap g may measure between about 0.02 millimeters and about 0.1 millimeters. As a non-limiting example, according to some embodiments, the width w of the gap g may measure about 50 micrometers. According to some embodiments, a thickness q of window element 786b may measure between about 0.2 millimeters and about 1 millimeter. As a non-limiting example, according to some embodiments, the thickness q of window element 786b may measure about 0.6 millimeters.

It is to be understood that the above description of the affixing of window element 786b within side-hole 705b, and, in particular, the geometry and dimensions of the window element and the side-hole, may apply not only for the rest of side-holes for the side-illumination modules (side-holes—not shown in FIG. 7—which are specific embodiments of side-holes 305c, and side-holes 303b and 303c), but also for the front holes for the front illumination modules (front holes—not shown in FIG. 7—which are specific embodiments of front holes 301b, 301c, and 301d). Further, according to some embodiments, the above description also applies for the holes for the cameras (front holes and side-holes—not shown in FIG. 7—which are specific embodiments of front hole 301a, and side-holes 303a and 305a).

As depicted in FIG. 7, side-hole 705b may include a support ledge 765b configured to support (sustain) window element 786b within side-hole 705b prior to the soldering of window element 786b onto the rim of side-hole 705b. According to some embodiments, support ledge 765b may extend along the full length of the rim of side-hole 705b and centrally project therefrom (i.e. project towards the center of the circle defined by side-hole 705b). According to some alternative embodiments, support ledge 765b may be segmented. That is, support ledge 765b may include at least two separate segments, with each of the segments projecting centrally from the rim of side-hole 705b. Each of the rest of the side-holes for the side-illumination modules may include essentially identical or similar support ledges. Further, according to some embodiments, each of the front holes for the front illumination modules may include essentially identical or similar support ledges. According to some embodiments, each of the holes for the cameras may include a support ledge with full circumferential support (i.e. the support ledge is not segmented), thereby preventing “parasitic” light from reaching the respective image sensor.

According to some embodiments, segmented support ledges may allow for further compactification of the arrangement of the camera and the illumination modules within the tip component, More specifically, since the “missing segments” of the support ledge do not block light emitted by the respective illumination module, these “missing segments” therefore allow for increased illumination by the illumination module, and therefore allow using a smaller illumination module to achieve a desired illumination level.

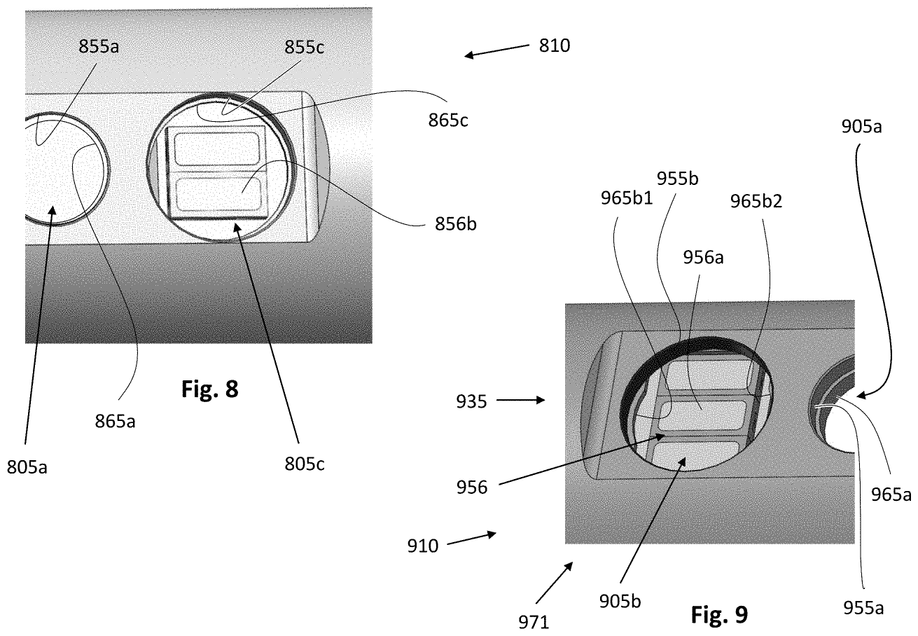

FIG. 8 provides a schematic side-view of a portion of a tip component 810, according to some embodiments. Tip component 810 is a specific embodiment of tip component 210. According to some embodiments, tip component 810 is a specific embodiment of tip component 310. According to some embodiments, tip component 810 is a specific embodiment of tip component 610. According to some embodiments, tip component 810 is a specific embodiment of tip component 710.

Depicted are a (central) side-hole 805a for a side-camera (not shown) and a side-hole 805c for a side-illumination module 856c, according to some embodiments. Side-hole 805a includes a support ledge 865a projecting centrally (towards the center of the circle defined by side-hole 805a) from a rim 855a of side-hole 805a. Support ledge 865a may constitute a circular flange extending centrally along the full circumference of side-hole 805a. Similarly, side-hole 805c includes a support ledge 865c projecting centrally (towards the center of the circle defined by side-hole 805c) from a rim 855c of side-hole 805c. Support ledge 865c may constitute a circular flange extending along the full circumference of side-hole 805c. As a non-limiting example, in FIG. 8 side-illumination module 856b is depicted as including two LEDs.

Each of the remaining holes for the cameras (both side and front) and the illumination modules (both side and front) may include essentially identical or similar support ledges to support ledge 865a and support ledge 865c, respectively.

FIG. 9 provides a schematic perspective side-view of a portion 971 of a hull 935 of a tip component 910, according to some embodiments. Tip component 910 is a specific embodiment of tip component 210. According to some embodiments, tip component 910 is a specific embodiment of tip component 310. According to some embodiments, tip component 910 is a specific embodiment of tip component 610. According to some embodiments, tip component 910 is a specific embodiment of tip component 710.

Depicted are a central side-hole 905a for a side-camera (not shown) and a side-hole 905b for a side-illumination module 956a of a second side-illumination unit 956, according to some embodiments. Second side-illumination unit 956 is a specific embodiment of second side-illumination unit 256. Side-hole 905a includes a support ledge 965a projecting centrally (towards the center of the circle defined by side-hole 905a) from a rim 955a of side-hole 905a. Support ledge 965a may constitute a circular flange extending centrally along the full circumference of side-hole 905a, essentially as described above with respect to side-hole 805a of tip component 810. Side-hole 905b includes a support ledge 965b projecting centrally (towards the center of the circle defined by side-hole 905b) from a rim 955b of side-hole 905b. Support ledge 965b may include two opposite-facing, or substantially opposite-facing, segments: a segment 965b1 and a second segment 965b2. As a non-limiting example, in FIG. 9 side-illumination module 956a is depicted as including three LEDs.

According to some embodiments, support ledge 965b may extend along less than two thirds of the circumference of the respective rim. For example, each of first segment 965b1 and second segment 965b2 may extend along less than 33% of the circumference of a rim 955b of side-hole 905b. According to some embodiments, support ledge 965b may extend along less than half of the circumference of the respective rim. For example, each of first segment 965b1 and second segment 965b2 may extend along less than 25% of the circumference of rim 955b of side-hole 905b.

Each of the remaining holes for the cameras may include essentially identical or similar support ledges to support ledge 965a. Each of the remaining holes for the illumination modules (both side and front) may include essentially identical or similar support ledges to support ledge 965b, respectively.

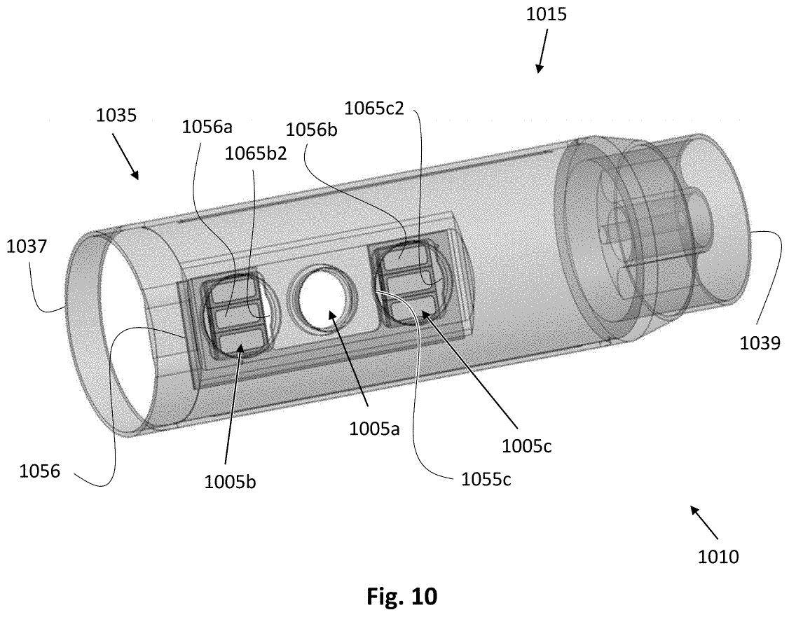

FIG. 10 provides a schematic, perspective, semi-transparent view of a hull 1035 of a tip third part 1015 (not all parts of which are shown) of a tip component 1010 (not all parts of which are shown). Tip component 1010 is a specific embodiment of tip component 910. Tip third part 1015 includes a second side-illumination unit 1056 installed thereon, according to some embodiments. Second side-illumination unit 1056 is a specific embodiment of second side-illumination unit 956.

Depicted is a second side-holes arrangement 1005, according to some embodiments. Side-holes arrangement 1005 includes a central side-hole 1005a for a second side-camera (not shown) centrally positioned between two side-holes 1005b and 1005c for side-illumination modules 1056a and 1056b (of side-illumination unit 1056), respectively. Side-hole 1005b is positioned distally to central side-hole 1005a while side-hole 1005c is positioned proximally to central side-hole 1005a. That is, side-hole 1005b is positioned more closely to a hull distal end 1037 of hull 1035 than each of side-holes 1005a and 1005c, and side-hole 1005c is positioned more closely to a hull proximal end 1039 of hull 1035 than each of side-holes 1005a and 1005b. Each of side-holes 1005b and 1005c may include a segmented support ledge: segmented support ledges 1065b and 1065c, respectively.

More specifically, and as shown in FIG. 10, support ledge 1065b may include two opposite-facing, or substantially opposite-facing, segments: a (distal) first segment (not visible in FIG. 10) and a (proximal) second segment 1065b2. Similarly, support ledge 1065c may include two opposite-facing, or substantially opposite facing, segments: a (distal) first segment (not visible in FIG. 10) and a (proximal) second segment 1065c2. According to some embodiments, each of the support ledges may extend along less than two thirds of the circumference of the respective rim. For example, each of the first segment of support ledge 1065c and second segment 1065c2 may extend along less than 33% of the circumference of a rim 1055c of side-hole 1005c. According to some embodiments, each of the support ledges may extend along less than half of the circumference of the respective rim. For example, each of the first segment of support ledge 1065c and second segment 1065c2 may extend along less than 25% of the circumference of rim 1055c of side-hole 1005c. Each of the remaining holes for the illumination modules (both side and front) may include essentially identical or similar support ledges to support ledges 1065b and 1065c.

FIG. 11A schematically depicts a front holes arrangement 201 on front surface 222, according to some embodiments. Front holes arrangement 201 includes a central front hole 201a and, according to some embodiments, three surrounding front holes 201b, 201c, and 201d. Front holes 201b, 201c, and 201d may be symmetrically disposed around front hole 201a. Indicated is a diameter D of front surface 222 (which according to some embodiments equals the diameter of a tip component 210), a characteristic scale d_{F} of front hole 201a and a characteristic scale d′_{F} of front holes 201b, 201c, and 201d. In embodiments wherein the front holes are round, the characteristic scales d_{F} and d′_{F} correspond to the respective diameters of front hole 201a and front holes 201b, 201c, and 201d, respectively. Also indicated is a spacing y_{F} between a rim of front hole 201a and rims of front holes 201b, 201c, and 201d, a distance v_{F} between the centers of front holes 201a and 201b (which may equal the distance between the centers of front holes 201a and the centers of front holes 201c and 201d), and a distance t between the rims of the surrounding front holes 201b, 201c, and 201d and the rim of front surface 222.

According to some embodiments, a relation between the diameter D, the characteristic scale d_{F}, and the spacing y_{F} may be related by y_{F}≈(D−3·d_{F})/4. Here, the symbol ≈ is used to indicate that the left-hand side of an equation is about equal to the right-hand side of the equation. More generally, the magnitude of y_{F} may be given by (a·D−b·d_{F})/c, wherein a equals about 1, b equals about 3, and c equals about 4. According to some embodiments, a magnitude of the distance t may be about equal to the magnitude of the spacing y_{F}. According to some embodiments, the spacing y_{F} and/or the distance t may be as small as about 0.05 millimeters.

FIG. 11B schematically depicts first side-holes arrangement 203 set within first side-niche 274, according to some embodiments. First side-holes arrangement 203 includes a central first side-hole 203a and, according to some embodiments, a pair of first side-holes 203b and 203c respectively positioned distally and proximally to first side-hole 203a. That is, side-hole 203b is positioned more closely to front surface 222 than each of first side-holes 203a and 203c, and first side-hole 203c is positioned more closely to shaft body 208 than each of first side-holes 203a and 203b. Indicated is a characteristic scale d_{S} of first side-hole 203a and a characteristic scale d′_{S} of first side-holes 203b and 203c. In embodiments wherein the first side-holes are round, the characteristic scales d_{S} and d′_{S} correspond to the respective diameters of first side-hole 203a and first side-holes 203b and 203c, respectively. Also indicated is a spacing y_{S} between a rim of first side-hole 203a and rims of first side-holes 203b and 203c, and a distance vs between the centers of first side-holes 203b and 203c.

FIG. 11C schematically depicts second side-holes arrangement 205 set within second side-niche 276, according to some embodiments. Second side-holes arrangement 205 includes a central second side-hole 205a and, according to some embodiments, a pair of second side-holes 205b and 205c respectively positioned distally and proximally to second side-hole 205a. That is, side-hole 205b is positioned more closely to front surface 222 than each of first side-holes 205a and 205c, and first side-hole 205c is positioned more closely to shaft body 208 than each of first side-holes 205a and 205b. Indicated is a characteristic scale d″_{S} of second side-hole 205a and a characteristic scale d′″_{S} of second side-holes 205b and 205c. In embodiments wherein the second side-holes are round, the characteristic scales d″_{S} and d′″_{S} correspond to the respective diameters of second side-hole 205a and second side-holes 205b and 205c, respectively. Also indicated is a spacing y′_{S} between a rim of second side-hole 205a and rims of second side-holes 205b and 205c, and a distance v′_{S} between the centers of second side-holes 205b and 205c.

According to some embodiments, the characteristic scale d″_{S} may be equal to, or at least comparable to, the characteristic scale d_{S}, the characteristic scale d′″_{S} may be equal to, or at least comparable to, the characteristic scale d′_{S}, and the spacing y′_{S} may be equal to, or at least comparable to, the spacing y_{S}.

As a non-limiting example, according to some embodiments, each of front holes 201 and side-holes 203 and 205 is round, 3.2 mm≤d_{F}≤4.9 mm, each of d′_{F}, d_{S}, and d″_{S} is comparable to d_{F}, d′_{S} and d′″_{S} each range from d_{F} to about 1.3·d_{F}, 10 mm≤D≤15 mm, y_{F} may be equal to about 0.05 millimeters, y_{S} and y′_{S} may each range from about 0.5 millimeters to about 1.25 millimeters, and t may be comparable to y_{F}.

As another non-limiting example, according to some embodiments, each of front holes 201 and side-holes 203 and 205 is round, 0.1 mm≤d_{F}≤0.4 mm, each of d′_{F}, d_{S}, and d″_{S} is comparable to d_{F}, d′_{S} and d′″_{S} each range from about d_{F} to about 1.3·d_{F}, 2 mm≤D≤6 mm, y_{F} may be equal to about 0.05 millimeters, y_{S} and y′_{S} may each range from about 0.5 millimeters to about 1.25 millimeters, and t may be comparable to y_{F}.

As yet another non-limiting example, according to some embodiments, each of front holes 201 and side-holes 203 and 205 is round, d_{F} is equal to about 3.4 millimeters, each of d′_{F}, d_{S}, and d″_{S} is comparable to d_{F}, d′_{S} and d′″_{S} are each equal to about 4.5 millimeters, v_{F} is equal to about 3.7 millimeters, v_{S} and v′_{S} each equal about 10.4 millimeters, y_{F} may be equal to about 0.3 millimeters, y_{S} and y′_{S} may each range from about 0.5 millimeters to about 1.25 millimeters, and t may be comparable to y_{F}.

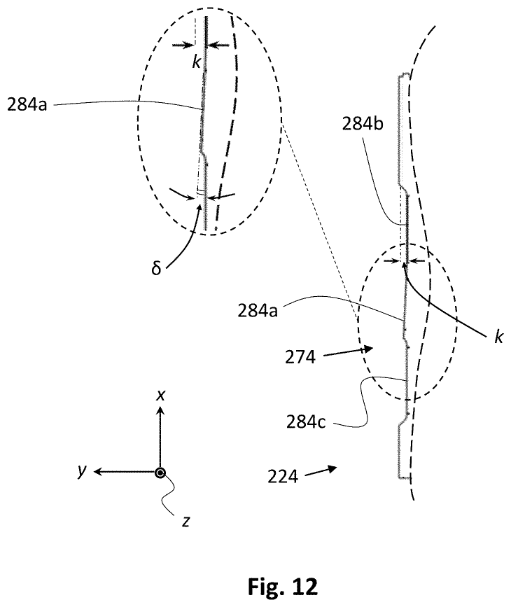

FIG. 12 provides a schematic, longitudinal, cross-sectional view of a first side-section of tip component 210, according to some embodiments. The first side-section includes first side-surface 224. The cross-section is taken along the same line as in FIG. 2C. First side-niche 274 is depicted. According to some embodiments, the optical axis O₁ of first side-lens assembly 264 (not shown in FIG. 12) may be slightly tilted relative to the positive y-axis. In such embodiments, window element 284a may be slightly offset (tilted) with respect to the x-axis. (Each of window elements 284b and 284c may be parallel to the x-axis.) An angle δ indicates the offset angle of window element 284a relative to the x-axis.

According to some embodiments, δ is smaller than about 5 degrees. According to some embodiments, δ is smaller than about 3 degrees. According to some embodiments, δ is smaller than about 2 degrees. Each option corresponds to different embodiments.

Also indicated is a depth k of first side-niche 274. According to some embodiments, the depth k may be between about 0.01 millimeters and about 1 millimeter, between about 0.05 millimeters and about 1 millimeter, or between about 0.1 millimeters and about 1 millimeter. Each possibility corresponds to separate embodiments.

It will be understood that the scope of the disclosure also covers shafts for semi-rigid endoscopes. As used herein, according to some embodiments, a “semi-rigid endoscope” may refer to an endoscope including a semi-rigid shaft. The semi-rigid shaft may include a rigid elongated member, a distal tip portion, and a maneuvering portion mounted between, and mechanically coupling, the elongated member and the distal tip portion, as described in PCT application publication No. WO2016181404 to A. Levy, which is incorporated herein by reference in its entirety. The semi-rigid shaft includes at least two cameras: a front camera and one or more side-cameras. The front camera is positioned on the distal tip portion. Each of the one or more side cameras may be positioned on the distal tip portion, the maneuvering portion, or the elongated member. The semi-rigid shaft further includes one or more illumination components configured to illuminate the FOV provided by the at least two cameras. The maneuvering portion is configured to bend, rotate, and/or angulate the distal tip portion, and thereby controllably change the combined FOV provided by the at least two cameras.

Thus, according to an aspect of some embodiments, not depicted in the figures, there is provided a semi-rigid endoscope. The semi-rigid endoscope may be similar to endoscope 100, but differs therefrom in including a semi-rigid shaft, as described in the preceding paragraph, instead of a rigid shaft. In particular, the cameras and illumination units of the semi-rigid endoscope may be similar to the cameras and illumination units of endoscope 100, or specific embodiments thereof, i.e. endoscopes 200 and 300, and endoscopes including tip components 610, 710, 810, 910, and 1010. Relative positions of the cameras and the illumination units may be similar, for example, to those of cameras 242, 244, and 246, and illumination units 252, 254, and 256, of endoscope 200. Window elements of the tip component may be affixed within holes on a housing of the tip component, essentially as described above with respect to endoscope 200 and endoscopes including tip component 710. In particular, the window elements may be made of, or include, sapphire glass while the solder material may include a noble metal, such as gold or platinum.

As used herein, according to some embodiments, the term “housing”, employed in reference to a tip component of an endoscope, refers to the tip component without windows (e.g. with the windows yet to be fitted on and affixed to the housing).

As used herein, according to some embodiments, the terms “circuit board” and “printed circuit board” are interchangeable.

As used herein, according to some embodiments, an element/component may be said to be made of a given material, when consisting of, or consisting essentially of, a composition including in weight at least 50%, 70%, 80%, or 90% of the given material. Each option corresponds to different embodiments.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1.-32. (canceled)

33. An elongated shaft for a multi-camera endoscope, the shaft comprising a shaft body and a tip component mounted on a shaft distal portion, the tip component comprises, accommodated within a housing of the tip component:
a front camera, a first side-camera, and a second side-camera positioned distally to the first side-camera, wherein the side-cameras face oppositely, or substantially oppositely, the cameras being configured to jointly provide a field-of-view (FOV) of at least about 270 degrees; and
a plurality of front illumination modules, two first side-illumination modules respectively proximally and distally positioned relative to the first side-camera, and two second side-illumination modules respectively proximally and distally positioned relative to the second side-camera, the illumination modules being configured to jointly illuminate the FOV;
wherein the tip component comprises a plurality of window elements of sapphire glass, which are soldered, using a noble-metal as solder material, within respective holes on the housing, each of the cameras and illumination modules being positioned behind a respective window element from the plurality of window elements.

34. The elongated shaft of claim 33, wherein the noble metal comprises gold and/or platinum.

35. The elongated shaft of claim 33, wherein rims of the holes comprise stainless steel and wherein the window elements are soldered onto the rims.

36. The elongated shaft claim 33, wherein each of the window elements is set on a respective support ledge extending from a rim of the respective hole, and wherein the support ledges of at least some of the window elements, associated with the illumination modules, are segmented, thereby allowing provision of increased illumination by the illumination modules.

37. The elongated shaft of claim 36, wherein segments pertaining to each of the segmented support ledges together circumferentially constitute less than 50% of a perimeter of the respective rim; and/or wherein the segments pertaining to each segmented support ledge consist essentially of four segments at most.

38. The elongated shaft of claim 33, wherein a diameter D of the tip component measures between about 2 millimeters and about 15 millimeters.

39. The elongated shaft of claim 38, wherein a characteristic scale d_{F} of the window element of the front camera measures between about 20% to about 50% of the diameter D of the tip component.

40. The elongated shaft of claim 39, wherein the plurality of front illumination modules comprises three illumination modules;
wherein a front surface of the housing is flat, or substantially flat, and comprises the window element of the front camera and three additional window elements, each of the additional window elements having positioned behind thereto one of the front illumination modules, respectively;
wherein a relation between the diameter D, the characteristic scale d_{F}, and a spacing y_{F} between the window element of the front camera and nearest window element from the additional window elements is given by y_{F}≈(D−3·d_{F})/4.

41. The elongated shaft claim 40, wherein each of the additional window elements has a respective characteristic scale which is comparable to the characteristic scale d_{F}.

42. The elongated shaft of claim 33, wherein respective diameters of the window elements of the first and second side-cameras measure between about 2.5 millimeters and about 5 millimeters, and wherein respective diameters of the window elements of the first and second side-illumination modules measure between about 3.5 millimeters and about 5.5 millimeters, and wherein respective distances between each of the window elements of the side-cameras and the two window elements of the illumination modules, which are adjacent thereto, measure between about 0.5 mm and about 1.5 mm.

43. The elongated shaft of claim 33, wherein the window elements of the side-cameras have a smaller characteristic scale than the window elements of the side-illumination modules.

44. The elongated shaft of claim 33, wherein each of the window elements has a thickness measuring between about 0.2 millimeters and about 1 millimeter.

45. The elongated shaft of claim 33, wherein the gold-soldering between each of the window elements and the rim of the respective hole fluidly seals a gap between the window element and the rim, and wherein the fluid-sealing provided by the gold-soldering is configured to withstand autoclave sterilization.

46. The elongated shaft of claim 33, wherein optical axes of the cameras span a plane comprising a longitudinal axis of the elongated shaft.

47. The elongated shaft of claim 33, wherein the housing comprises a first part, a second part, and a third part;
wherein the first part comprises the front surface, the front camera, and the plurality of front illumination modules;
wherein the second part comprises a cover section, comprising the holes of the first side-camera and first side-illumination modules, and the first side-camera and first side-illumination units;
wherein the third part comprises a hull, the second side-camera and second side-illumination units;
wherein the hull is open on a distal end thereof and a proximal end thereof, and comprises an elongated opening on a first side-surface thereof, on which the cover section is configured to be fitted, and, on second side-surface thereof, the holes of the second side-camera and the second side-illumination modules; and
wherein the hull is configured to accommodate the cameras and the illumination modules.

48. The elongated shaft of claim 47, wherein each of the first part and second part is soldered, welded, and/or glued to the third part.

49. The elongated shaft of claim 33, wherein each of the illumination modules comprises one or more light-emitting diodes (LEDs).

50. A method for affixing windows on a shaft of a multi-camera endoscope, the method comprising:
providing a housing of a tip component of a shaft of a multi-camera endoscope, the housing comprising at least a pair of holes arrangements, each of the holes arrangements comprising at least two holes for a window element for a camera and for at least one window element for at least one illumination module;
for each of the holes:
fitting a respective window element on a support ledge extending centrally from a rim of the hole; and
soldering the window element onto the rim of the hole, thereby affixing the window element onto the housing;
wherein the support ledges of at least some of the illumination module window elements are segmented, thereby allowing provision of increased illumination by the illumination modules.

51. The method of claim 50, wherein the at least some of the window elements comprise sapphire glass and wherein at least some of the window elements are soldered onto the rim of the respective hole using solder material comprising a noble metal, said noble metal comprises gold and/or platinum.

52. The method of claim 50, wherein the soldering fluidly seals gaps between the window elements and the rims of the holes and is configured to withstand autoclave sterilization.
